(19) United States
(12) Patent Application Publication
(10) Pub. No.: US 2006/0094017 A1
(43) Pub. Date: May 4, 2006

(54) Immunogens for hiv vaccine

(76) Inventors: Anthony J. Conley, RAHWAY, NJ (US); Beverly H. Galinski, Doylestown, PA (US); Allison Montalvo, Phoenixville, PA (US)

Correspondence Address: MERCK AND CO., INC, P O BOX 2000, RAHWAY, NJ, 07065-0907, US

(21) Appl. No.: 10/544,499

(22) PCT Filed: Feb. 10, 2004
(86) PCT No.: PCT/US04/05821

§ 371 Date: Aug. 3, 2005

Related U.S. Application Data
(60) Provisional application No. 60/447,590, filed on Feb. 14, 2003.

(51) Int. Cl. C12Q 1/70; C12Q 1/68

(52) U.S. Cl. 435/6; 435/5

(57) ABSTRACT

Peptidyl sequences, called mimotopes, are disclosed which mimic the binding site of the broadly neutralizing human monoclonal antibody, 2G12, specific for the HIV protein gp120. The mimotopes are identified from a chimeric protein III (pIII) phage display library, each phage containing an additional random 15 amino acids near the N-terminus of pIII. Immunological conjugates of HIV-specific mimotopes that are useful for vaccination against HIV infection are disclosed. Methods for using the mimotopes and their immunological conjugates as part of an HIV vaccine regime, as well as diagnostic tools to perform viral assays, are also disclosed.

### CROSS-REFERENCE TO RELATED APPLICATIONS

The present application claims the benefit of U.S. Provisional Application No. 60/447,590, filed Feb. 14, 2003, hereby incorporated by reference herein.

### STATEMENT REGARDING FEDERALLY-SPONSORED R&D

Not Applicable

### REFERENCE TO MICROFICHE APPENDIX

Not Applicable

### FIELD OF THE INVENTION

The present invention relates to peptide sequences, called mimotopes, that bind to a broadly neutralizing human monoclonal antibody specific for the HIV protein gp120, 2G12. The present invention also relates to immunological conjugates of HIV-specific mimotopes, as well as methods for using the mimotopes and their immunological conjugates as part of an HIV vaccine and as a diagnostic tool to perform viral assays.

### BACKGROUND OF THE INVENTION

Acquired Immune Deficiency Syndrome (AIDS) is the clinical manifestation of the infection of CD4 helper T-cells and other cell targets by human immunodeficiency virus (HIV). AIDS is characterized by opportunistic infections and certain malignancies.

HIV has a unique collection of attributes: HIV targets the immune system; it possesses a reverse transcriptase capable of turning out highly mutated progeny; it is sequestered from the immune system; and it has hypervariable sequences in the (env) region. See, e.g., Hilleman, 1988, Vaccine 6:175-179 and Barnes, 1988, Science 240:719-721. One consequence of these attributes is the diversity of HIV serotypes.

Elicitation of neutralizing antibodies capable of reacting with HIV primary isolates is regarded as one of the key consequential features in the successful design of an HIV immunological therapy. The term “neutralizing” as applied to antibodies means that viral exposure to such antibodies, whether in vitro or in vivo, results in the attenuation or abrogation of any or all of the recognized virus-mediated pathophysiologic functions characteristic of HIV infection and disease including cellular fusion, cellular infection, CD4 receptor bearing cell depletion, and viral proliferation. Neutralizing antibodies meeting these criteria have been detected in the sera of HIV-infected patients. Identifying one or more immunogens that will elicit neutralizing antibodies is a difficult aspect of developing a preventative vaccine for HIV infection. What is needed are immunogens that are capable of generating a response that is sufficient to neutralize primary isolates and be broadly reactive. However, immune sera generated in studies with immunogens based on HIV antigens often fail to neutralize primary isolates and are frequently extremely type-specific.

As an alternative, both specific, virus-neutralizing and broadly-reactive, anti-HIV monoclonal antibodies may provide clues to help identify immunogens with the potential of eliciting neutralizing antibodies. By identifying the binding determinants for these known anti-HIV antibodies, one can focus future antibody generation, as part of an HIV immunological therapy, on only those epitopes necessary for successful vaccination. The present invention addresses and meets these needs by disclosing peptide sequences, called mimotopes, that bind to the virus-neutralizing monoclonal antibody, 2G12. The antibody 2G12 is broadly reactive in its ability to neutralize primary isolates and has demonstrated some protection in in vivo models of HIV infection. Thus, the incorporation of the identified peptidyl sequences in an immunogenic form in a vaccine could elicit a response similar to that of the original antibody.

### SUMMARY OF THE INVENTION

The present invention relates to peptides which mimic an antigenic epitope on the HIV protein gp120, a binding site of a neutralizing antibody. The peptides exemplified are synthetic peptidyl sequences selected out of a large, random phage display library based on their binding affinity to the known neutralizing monoclonal antibody, 2G12. Monoclonal antibody 2G12 is described further in Buchacher et al., 1994, AIDS Res. Hum. Retroviruses 10:359-369; and Trkola et al., 1996, J. Virol. 70:1100-1108, both of which are incorporated by reference. These peptides may be used as part of a preventative vaccine for HIV infection. Thus, one aspect of the present invention includes synthetic peptides useful as neutralization epitopes specific for gp120, known hereinafter as mimotopes.

The present invention further relates to mimotope peptides conjugated to an immunogenic carrier molecule. The mimotope and its carrier partner can be linked by non-specific cross-linking agents, monogeneric spacers or bigeneric spacers. Such immunological conjugates can be useful as HIV vaccines, generating novel antibodies which can neutralize HIV, and be part of active immunization therapies. The mimotopes of this invention and their immunological conjugates are also useful for diagnostic purposes as reagents in viral assays, such as those used to screen blood to determine if naturally occurring antibodies that bind the gp120 glycan structure are present.

The present invention further relates to an effective immunogen against HIV infection, and comprises an antigenic conjugate of formula I:
[mimotope(s)]ₙ˜[carrier]
wherein:

mimotope(s) are peptidyl sequences which mimic antigenic epitopes of gp120, said mimotope being a peptide comprising one or more amino acid sequences of Table A, (SEQ ID NOs:1-5);

n is 1-200, wherein n is the number of polypeptides of mimotope(s) covalently linked to a carrier;

˜ indicates covalent linkage; and

carrier is an immunogenic molecule to which the mimotope is conjugated, including, but not limited to, Neisseria meningitidis OMPC (Outer Membrane Proteosome Complex) particles, HBV-core antigen, HBV-surface antigen, immunogenic proteins such as tetanus or diphtheria toxoid or rotavirus VP6, other immunogenic glycoproteins such as HIV gp120 or gp41, and HIV capsid particles comprised of p24.

Mimotope peptides may exist alone as peptides, as internal sequences in proteins (e.g. phage pIII proteins), as part of an immunological conjugate with a carrier molecule, or as a fragment of a recombinant fusion protein with an immuno-enhancer sequence. The position of the mimotope in a fusion protein may be N-terminal, internal or C-terminal.

The present invention relates to methods of using the mimotopes disclosed herein as part of a vaccine for the prevention of HIV infection. An effect of mimotope-related vaccines should be a lower transmission rate to previously uninfected individuals (i.e., prophylactic applications) and/or reduction in the levels of viral loads within an infected individual (i.e., therapeutic applications), so as to prolong the asymptomatic phase of HIV infection.

The present invention further relates to methods of using the mimotopes disclosed herein as part of a diagnostic tool to perform anti-viral antibody assays, such as those used to screen blood.

The terms “protein,” “peptide,” “oligopeptide,” and “polypeptide” and their plurals have been used interchangeably to refer to chemical compounds having amino acid sequences of five or more amino acids.

When any variable occurs more than one time in any constituent or in Formula I (e.g. mimotope), its definition on each occurrence is independent of its definition at every other occurrence. Also, combinations of substituents and/or variables are permissible only if such combinations result in stable compounds.

### DETAILED DESCRIPTION OF THE INVENTION

The present invention relates to randomly generated peptide sequences which mimic antigenic epitopes on the HIV protein gp120, a binding site of a neutralizing antibody, known hereinafter as mimotopes. The peptides exemplified are synthetic peptidyl sequences selected out of a large, random phage display library based on their binding affinity to the known HIV neutralizing monoclonal antibody, 2G12. Mimotope peptides may exist alone as peptides, as internal sequences in proteins (e.g. phage pIII proteins), as part of an immunological conjugate with a carrier molecule, or as a fragment of a recombinant fusion protein with an immuno-enhancer sequence. The position of the mimotope in a fusion protein may be N-terminal, internal or C-terminal. These peptides may be used as part of an anti-HIV vaccine, as well as being useful for diagnostic purposes as reagents in viral assays.

The mimotopes of the present invention bind to monoclonal antibody (Mab) 2G12, an HIV broadly-neutralizing antibody that binds gp120. Mab 2G12 recognizes an epitope located around the C4/V4 region of gp120. More specifically, Mab 2G12 binding to gp120 is dependent on the mannose residues in N-linked high-mannose and/or hybrid glycan chains within the 2G12 epitope (Sanders et al., 2002, J. Virol. 76:7293-7302; Scanlan et al, 2002, J. Virol. 76:7306-7321). The mimotopes of the present invention were selected and identified from an oligopeptide epitope library. The sequences of these polypeptides were deduced from their corresponding DNA sequences. The mimotope peptides of the present invention are characterized as having the sequences disclosed in Table A, SEQ ID NOs:1-5, below.

TABLE A
Mimotopes Identified by Selection with Mab 2G12

| | | |
|---|---|---|
| 1. | RSGHKVWVVSTKESS | (SEQ ID NO:1) |
| 2. | KCCFAESSRSGTGRY | (SEQ ID NO:2) |
| 3. | WKIPDHGIVVFSWFP | (SEQ ID NO:3) |
| 4. | VLRLMECHFQCVPSL | (SEQ ID NO:4) |
| 5. | TLKSLPYRAVLGAQA | (SEQ ID NO:5) |

Phage epitope libraries are unusually versatile vehicles for identifying new antigens or ligands. Generally, a small, randomly generated DNA sequence, e.g., 45 base pairs, which will generate exposed oligopeptide surfaces in the mature phage, is inserted into a phage genome. The mature phage are mixed with a screening antibody of desired specificity. The bound phage are separated away from unbound phage, cloned, and sequenced. A conventional example of a phage epitope library is the filamentous phage fd and its gene III coding for minor coat protein pIII. See, e.g., Parmley et al., 1988, Gene 73:305-318; and Scott et al., 1990, Science 249:386-390, which set forth extensive discussion and detail on construction of these libraries.

In an exemplified method, the mimotopes of the present invention were identified by screening a chimeric protein III (pIII) phage display library with Mab 2G12 using a three-cycle panning procedure. The library used is characterized in Keller et al., 1993, Virology 123:709-716, and displays randomly generated epitope polypeptides that are accessible to the screening antibody. A polystyrene bead coated with Mab 2G12 was incubated in solution with the phage library (about 1×10¹⁰ to 1×10¹¹ phage particles). Those phage containing mimotope peptides that recognize the 2G12 antibody adhered to the bead. After extensive washing, the bound phage were then dislodged from the bead. A small portion of the dislodged phage were used to infect E. coli for plating purposes. Individual phage-transduced colonies were selected for phage isolation and sequencing of the chimeric region of pIII. These phage represent those identified from round one of the panning procedure, yielding round one mimotope peptides. The remainder of the dislodged phage from round one was used to produce a small, unselected E. coli culture, referred to as the round two pool. The round two pool was screened by the same procedure described above, re-selecting those phage (and their mimotope sequences) that bound to Mab 2G12 in round one, and generating round two phage and their resulting mimotopes. The cycle was repeated once more, generating round three phage and mimotopes. Sequencing of the inserts was accomplished using primers specific to the chimeric region of pIII. The binding of these sequences was then confirmed by surface plasmon resonance (SPR) use BIACore technology. The mimotopes disclosed in the instant application represent those sequences identified from the round three pool of enriched phage.

Large amounts of DNA coding for mimotope peptides may be obtained using PCR amplification techniques as described in Mullins et al., U.S. Pat. No. 4,800,159 and Innis et al., 1990, PCR Protocols Academic Press, both of which are hereby incorporated by reference. Once the DNA sequence is determined, its amino acid sequence can be deduced by translating the DNA sequence. The resulting amino acid sequence representing the mimotope of the HIV envelope gene can be synthesized in large quantities by either organic synthesis or recombinant expression.

Long peptides may be synthesized on solid-phase supports using an automated peptide synthesizer as described by Kent et al., 1985, “Modem Methods for the Chemical Synthesis of Biologically Active Peptides”, Alitalo et al. (Eds.), Synthetic Peptides in Biology acid Medicine, Elsevier pp. 29-57, which is hereby incorporated by reference. Manual solid-phase synthesis may be performed as described, for example, in Merrifield, 1963, Am. Chem. Soc. 85:2149, which is hereby incorporated by reference, or known improvements thereof. Solid-phase peptide synthesis may also be performed by the Fmoc method, which employs very dilute base to remove the Fmoc protecting group. Solution-phase synthesis is usually feasible only for selected smaller peptides. For preparing cocktails of closely related peptides, see, e.g., Houghton, 1985, Proc. Natl. Acad. Sci. USA 82:5131.

The mimotope gene may be recombinantly expressed by molecular cloning into an expression vector (such as pcDNA3.neo, pcDNA3.1, pCR2.1, pBlueBacHis2 or pLITMUS28) containing a suitable promoter and other appropriate transcription regulatory elements, and transferred into prokaryotic or eukaryotic host cells to produce the mimotope peptide. Expression vectors are defined herein as DNA sequences that are required for the transcription of cloned DNA and the translation of their mRNAs in an appropriate host. Such vectors can be used to express recombinant DNA in a variety of recombinant hosts cells such as bacteria, yeasts, blue green algae, plant cells, insect cells and mammalian cells. An appropriately constructed expression vector should contain: an origin of replication for autonomous replication in host cells, selectable markers, a limited number of useful restriction enzyme sites, a potential for high copy number, and active promoters. A promoter is defined as a DNA sequence that directs RNA polymerase to bind to DNA and initiate RNA synthesis. A strong promoter is one which causes mRNAs to be initiated at high frequency. Techniques for such manipulations can be found in Sambrook et al., 1989,

Molecular Cloning: A Laboratory Manual, Cold Spring Harbor Laboratory, Cold Spring Harbor, N.Y., and are well known and available to an artisan of ordinary skill in the art.

Expression vectors may include, but are not limited to, cloning vectors, modified cloning vectors, specifically designed plasmids or viruses. Commercially available mammalian expression vectors may be suitable for recombinant mimotope expression. Also, a variety of commercially available bacterial, fungal cell, and insect cell expression vectors may be used to express recombinant mimotopes in the respective cell types.

Recombinant host cells may be prokaryotic or eukaryotic, including but not limited to bacteria such as E. coli, fungal cells such as yeast, mammalian cells such as cells lines of bovine, porcine, monkey, and rodent origin, and insect cells.

The mimotope gene of this invention comprises any DNA encoding a mimotope of Table A. The mimotope gene may also include other features such as a promoter and/or operator, ribosome binding sites, termination codons, enhancers, terminators, or replicon elements. These additional features can be inserted into the vector at the appropriate site(s) by conventional splicing techniques.

The expression vector containing the appropriate gene coding for a mimotope peptide may be introduced into host cells via any one of a number of techniques, including but not limited to transformation, transfection, protoplast fusion, and electroporation. The expression vector-containing cells are individually analyzed to determine whether they produce the mimotope of interest. Identification of mimotope expressing cells may be done by several means, including but not limited to immunological reactivity with anti-mimotope antibodies.

Recombinant mimotope may possess additional and desirable structural modifications not shared with the same organically synthesized peptide, such as adenylation, carboxylation, glycosylation, hydroxylation, methylation, phosphorylation or myristoylation. These added features may be chosen or preferred as the case may be, by the appropriate choice of recombinant expression system. On the other hand, a recombinant mimotope may have its sequence extended by the principles and practice of organic synthesis of described above.

As used herein, “purified” and “isolated” are utilized interchangeably to stand for the proposition that the nucleic acid, protein, or respective fragment thereof in question has been substantially removed from its in vivo environment so that it may be manipulated by the skilled artisan, such as but not limited to nucleotide sequencing, restriction digestion, site-directed mutagenesis, and subcloning into expression vectors for a nucleic acid fragment as well as obtaining the protein or protein fragment in pure quantities.

Following expression of a mimotope gene in a host cell, mimotope protein may be recovered. Several protein purification procedures are available and suitable for use: purification from cell lysates and extracts, or from conditioned culture medium, by various combinations of, or individual application of salt fractionation, ion exchange chromatography, size exclusion chromatography, hydroxylapatite adsorption chromatography and hydrophobic interaction chromatography. In addition, mimotope protein can be separated from other cellular proteins by use of an immunoaffinity column made with monoclonal or polyclonal antibodies specific for the mimotope protein.

The present invention further relates to the mimotope peptides disclosed in Table A conjugated to an immunogenic carrier molecule. The carrier molecule, usually a heterologous protein, can help to evoke an immune response. The mimotope and its carrier partner can be linked by non-specific cross-linking agents, monogeneric spacers or bigeneric spacers. Such immunological conjugates can be used as a component of a preventative vaccine for HIV infection, resulting in the generation of HIV-specific, broadly neutralizing antibodies for active immunity against HIV. The vaccine could be formulated with adjuvants known in the art, such as MPL-A, and adsorbed onto either Alum or aluminum phosphate. Alternatively, the mimotopes and their conjugates are also useful for the purpose of screening, clinical evaluation, and diagnosis of HIV as they may be part of assays used in the detection of HIV, or antibodies to HIV, in blood samples.

The present invention further relates to an effective immunogen against HIV infection, and comprises an antigenic conjugate of formula I:
[mimotope(s)]ₙ˜[carrier]
wherein:

mimotope(s) are peptidyl sequences which mimic antigenic epitopes of gp120, said mimotope being a peptide comprising one or more amino acid sequences of Table A, (SEQ ID NOs:1-5);

n is 1-200, wherein n is the number of polypeptides of mimotope covalently linked to a carrier;

˜ indicates covalent linkage; and

carrier is an immunogenic molecule to which the mimotope is conjugated.

In order to generate a useful vaccine, peptides, which are generally poorly immunogenic on their own, often must be conjugated to a carrier in a reproducible and quantifiable fashion. There are many candidate carrier molecules known in the art. See, e.g., Shodel et al., 1996, J. Biotechnol. 44:91-96; Lang and Korhonen, 1997, Behring Inst. Mitt. 98:400409; Brennan et al., 2001, Mol. Biotechnol. 17:15-26; Pumpens and Grens, 2201, Intervirology 44:98-114; and Simpson et al., 1999, Cell Mol. Life Sci. 56:47-61. Immunogenic carriers suitable for conjugation to the mimotopes of the present invention include, but are not limited to, N. meningitidis OMPC particles, HBV-core antigen, HBV-surface antigen, immunogenic proteins such as tetanus or diphtheria toxoid or rotavirus VP6, other immunogenic glycoproteins such as HIV gp120 or gp41, and HIV capsid particles comprised of p24. Each conjugate molecule of formula I may have different peptides conjugated thereto, or, alternatively, multiples of a single peptide species conjugated thereto, or a combination. The antigenic conjugates of this invention may also include T cell helper epitopes to effectuate a stronger helper T cell response, including but not limited to a synthetic, non-natural pan HLA DR-binding epitope (PADRE) (see, e.g., Alexander et al., 2000, J. Immunol., 164:1625-1633 and del Guercio et al., 1997, Vaccine 15:441-448).

The antigenic conjugates of this invention may be prepared by isolating, synthesizing and purifying their component parts (mimotope and carrier), and then conjugating mimotope and carrier together. Subsequent purification of conjugate mixtures may be performed as desired.

Antigenic conjugates of mimotope and a suitable immunogenic carrier have at least one covalent linkage between the component parts (i.e., mimotope and carrier), and typically have more than one mimotope molecule covalently bound to each carrier molecule. The mimotope and its carrier partner can be prepared separately, and then linked by non-specific cross-linking agents, monogeneric spacers or bigeneric spacers. Methods for non-specific cross-linking are well known in the art and include, but are not limited to, the following: reaction with glutaraldehyde; reaction with N-ethyl-N′-(3-dimethylaminopropyl)carbodiimide, with or without admixture of a succinylated carrier; periodate oxidation of glycosylated substituents followed by coupling to free amino groups of a protein carrier in the presence of sodium borohydride or sodium cyanoborohydride; diazotization of aromatic amino groups followed by coupling on tyrosine side chain residues of the protein; reaction with isocyanates; or reaction of mixed anhydrides. See, generally, Briand et al., 1985, J. Imm. Meth. 78:59-69.

In another embodiment of the invention, mimotope conjugates can be formed with a monogeneric spacer. These spacers are bifunctional and require functionalization of only one of the partners of the reaction pair to be conjugated before conjugation takes place. By way of illustration rather than limitation, an example of a monogeneric spacer involves coupling the mimotope peptide to one end of the bifunctional molecule adipic acid dihydrazide in the presence of carbodiimide. A diacylated hydrazine presumably forms with pendant glutamic or aspartic carboxyl groups of the mimotope. Conjugation is then performed by a second coupling reaction with carrier protein in the presence of carbodiimide. For similar procedures, see for example, Schneerson et al., 1980, J. Exp. Med. 152:361-376. Another example of a monogeneric spacer is described in Fujii et al., 1985, Int. J. Pept. Protein Res. 26:121-129.

In another embodiment of the invention, conjugates of mimotope and an immunogenic carrier can be formed with a bigeneric spacer. Bigeneric spacers are formed after each partner of the reaction pair to be conjugated, e.g., mimotope and carrier, is functionalized with a bifunctional spacer. Conjugation occurs when each functionalized partner is reacted with its opposite partner to form a stable covalent bond or bonds. See, for example, Marburg et al., 1986, J. Am. Chem. Soc. 108:5282-5287; and Marburg et al., U.S. Pat. No. 4,695,624, issued Sep. 22, 1987. Bigeneric spacers are preferred for preparing conjugates in human vaccines since the conjugation reaction is well characterized and easily controlled.

To evaluate mimotopes as immunogens, recombinant shuttle vectors coding for recombinant fusion polypeptides (RFPs) of novel mimotopes, such as pIII (with or without a polyhistidine tail), HBV-core antigen, HBV-surface antigen or protein A are made using known methods. Briefly, DNA sequences coding for a selected mimotope are ligated in-frame to DNA sequences coding for the fusion polypeptide. The resulting DNA fragment may be expressed in any of a wide variety of readily available recombinant expression systems, including Spodoptera frugiperda (Sf21) insect cells (Invitrogen) using a baculovirus vector (Pharmingen). In the alternative, the fusion peptides can be made by synthetic organic means, although this method is generally limited by feasibility and by practicality to smaller fusion peptides.

The present invention relates to methods of using the mimotopes disclosed herein as part of a vaccine for the prevention of HIV infection. The mimotope-related HIV vaccine, when administered alone or in combined modality and/or prime/boost regimen, will offer a prophylactic advantage to previously uninfected individuals and/or provide a therapeutic effect by reducing viral load levels within an infected individual, thus prolonging the asymptomatic phase of HIV infection.

The form of the antigen within the vaccine may take various molecular configurations. A single molecular species of the antigenic conjugate [mimotope(s)]ₙ˜[carrier] will often suffice as a useful and suitable antigen for the prevention or treatment of HIV infection. Other antigens in the form of cocktails are also advantageous, consisting of a mixture of conjugates that differ by the degree of substitution (n) or the amino acid sequences of the mimotope peptides, or both. An immunological vector or adjuvant may be added as a vehicle according to conventional immunological testing or practice.

The conjugates of this invention, when used as a vaccine, are administered in immunologically effective amounts. Dosages of between 10 μg and 500 μg of conjugate, and preferably between 50 μg and 300 μg of conjugate, are administered to a mammal to induce anti-mimotope, anti-HIV, or HIV-neutralizing immune responses. About two to four weeks after the initial administration, a booster dose may be administered, and then again whenever serum antibody titers diminish. The conjugate should be given intramuscularly at a concentration of between 10 μg/ml and 1 mg/ml, and preferably between 50 and 500 μg/ml, in a volume sufficient to make up the total required for immunological efficacy.

Adjuvants may or may not be added during the preparation of the vaccines of this invention. Alum is the typical and preferred adjuvant in human vaccines, especially in the form of a thixotropic, viscous, and homogeneous aluminum hydroxide gel. One embodiment of this invention is the prophylactic vaccination of patients with a suspension of alum adjuvant as vehicle and a cocktail of [mimotope(s)]ₙ˜[carrier] as the antigens.

In addition to using the mimotope peptides within an antigenic conjugate of formula I to elicit anti-HIV antibodies, the nucleic acid sequences encoding the mimotope peptides may be incorporated into gene sequences of immunogens, such as HBV-core antigen or HBV-surface antigen, and subsequently used as recombinantly expressed chimeric protein vaccines or as chimeric protein gene in a DNA vaccine. The chimeric gene construct may also be incorporated into an expression cassette for insertion into a recombinant viral vector, such as adenovirus serotype 5 vector. Vaccination may consist of mixed modalities. For example, a response may be elicited by first administering a DNA or an adenovirus construct expressing the chimeric mimotope-carrier construct followed by one or more doses of either the synthetic mimotope-conjugated product or the recombinantly produced chimeric mimotope-carrier product.

The vaccines of this invention may be effectively administered, whether at periods of pre-exposure and/or post-exposure, in combination with effective amounts of AIDS antivirals, immunomodulators, anti-infectives, or vaccines. Examples of AIDS antivirals are: Ganciclovir (DHPG, Cytovene®) from Hoffman-LaRoche (Nutley, N.J.); the HAART (Highly Active Anti-Retroviral Therapy) drugs including Indinavir from Merck (Rahway, N.J.), Melfinavir from Hoffman-LaRoche (Nutley, N.J.), Ritonavir from Abbott Laboratories (Abbott Park, Ill.) plus Saquinavir from Hoffman-La Roche (Nutley, N.J.), Ritonavir plus Indinavir from Merck (Rahway, N.J.), Ritonavir plus Lopinavir from Abbott Laboratories (Abbott Park, Ill.) or Efavirenz from Dupont Pharma (Wilmington, Del.) in combination with one of a number of other reverse transcriptase inhibitor combinations such as AZT plus 3TC both from GlaxoSmithKline (Philadelphia, Pa.) and AZT plus ddA from U.S. Bioscience Inc. (West Conshohocken, Pa.); d4T or ddI, both from Bristol-Myers Squibb (Princeton, N.J.); Foscarnet from AstraZeneca LP (Westborough, Mass.); ddC from Hoffman-La Roche; AZT or Alpha Interferon, both from GlaxoSmithKline (Philadelphia, Pa.); Rifabutin from Adria Laboratories (Dublin, Ohio); or Virazole from Viratek/ICN (Costa Mesa, Calif.).

Immunomodulators which can be combined with the vaccines of this invention include: Bropirimine from Pharmacia (Peapack, N.J.); Granulocyte Macrophage Colony Stimulating Factor from Genetics Institute (Boston, Mass.), Sandoz (E. Hanover, N.J.), Hoechst-Roussel (Somerville, N.J.), Immunex (Seattle, Wash.), or Schering Plough (Madison, N.J.); or IL-2 from Chiron (Emeryville, Calif.) and Hoffman-LaRoche (Nutley, N.J.).

Anti-infectives which can be used in combination with the vaccine of this invention include: Clindamycin with Primaquine from Pharmacia (Peapack, N.J.); Fluconazole from Pfizer (New York, N.Y.); Nystatin Pastille from Bristol-Myers Squibb (Princeton, N.J.); Pentamidine from LyphoMed (Rosemont, Ill.); Prirtexim from GlaxoSmithKline (Philadelphia, Pa.); Pentammidine isethionate from Fisons Corp. (Bedford, Mass.); Spiramycin from Rhone-Poulenc Pharmaceuticals (Princeton, N.J.); Intraconazole-R51211 from Janssen Pharmaceuticals (Piscataway, N.J.); or Trimetrexate from Pfizer (New York, N.Y.).

Other suitable compounds which can be used together with the vaccines of this invention include: Recombinant Human Erythropoietin from Ortho Pharmaceuticals (Raritan, N.J.) or Amgen (Thousand Oaks, Calif.); and Megestrol Acetate from Bristol-Myers Squibb (Princeton, N.J.).

It will be understood that the scope of combinations of the antigenic conjugates of this invention with AIDS antivirals, immunomodulators, anti-infectives or vaccines is not limited to those mentioned above, but includes in principle any combination with any pharmaceutical composition useful for the treatment of HIV or AIDS.

The present invention further relates to methods of using the mimotopes as part of a diagnostic tool to perform viral assays, such as those used to screen blood. The mimotopes and their immunological conjugates can be used to screen blood products for the presence of HIV antigen or HIV-specific antibodies. Thus, mimotopes and their immunological conjugates can be readily employed in a variety of immunological assays, including radioimmunoassay, competitive radioimmunoassay, enzyme-linked immunoassay, and the like. For an extensive discussion of these types of utilities, see, e.g., U.S. Pat. No. 5,075,211.

The following non-limiting Examples are presented to help illustrate the invention.

### EXAMPLE 1

### Bead Coating Procedure

Polystyrene beads of 0.25 inch diameter (Precision Plastic Ball Co., Franklin Park, Ill.) were disinfected for aseptic use by incubating overnight in 70% ethanol and rinsing several times with sterile water. To prepare for antibody coating, the beads were rinsed with 50 mM Na₂CO₃, pH 9.6, 0.02% sodium azide (Coating Buffer). The beads were incubated with 10 μg of the monoclonal antibody 2G12 in a 2 ml volume of Coating Buffer, with rocking, at 4° C. overnight The following day the antibody-coated beads were washed three times with phosphate buffered saline (PBS) and once with water. After washing, the antibody-coated beads were air dried and stored frozen at −20° C. until needed. Before use, the antibody-coated beads were coated with 10 mg/ml BSA (to block free sites on the plastic) in TTBS (50 mM Tris pH 7.5, 150 mM NaCl, 0.5% (v/v) Tween 20) for four or more hours.

### EXAMPLE 2

### Stringent Phage Selection with Antibody-Coated Beads

The process used to identify the mimotopes of the present invention consists of a three-cycle panning procedure. Generally, an antibody-coated bead is incubated in solution with a phage library (about 1×10¹⁰ to 1×10¹¹ phage particles) displaying potential mimotope peptides. Those phage containing mimotope peptides that recognize the target antibody adhere to the bead. The bead is then removed from the solution, washed extensively, and the bound phage are dislodged from the bead. A small portion of the recovered phage (round one phage) is then used to infect E. coli for plating. Individual phage-transduced colonies are selected to harvest phage and for sequencing of the chimeric region of pIII. This is round one of the panning procedure, yielding round one mimotope peptides. The remainder of the round one pool of phage is used to generate an unselected E. coli culture, referred to as the round two pool, to be incubated with a fresh, antibody coated bead. This second round of panning re-selects those phage that bound to the antibody in round one of the procedure, generating round two phage and their resulting mimotopes. The cycle is repeated once more to generate round three phage and mimotopes. Sequencing of the inserts is accomplished using primers specific for the chimeric region of pIII. The binding of these sequences is then confirmed by an independent method, such as by surface plasmon resonance (SPR), i.e. by BIACore analysis. The mimotopes disclosed in the instant application represent those sequences identified from the round three pool of enriched phage.

The mimotopes of the present invention were selected from a chimeric protein III (pIII) phage display library which displays any one of about 100×10⁶ chimeric pIII, each with an additional random 15 amino acids near the N-terminus of the pIII. The library used is characterized in Keller et al., 1993, Virology 123:709-716. One polystyrene bead previously coated with Mab 2G12 was washed in TTBS and then resuspended into 1 ml TTBS, 1 mg/ml BSA and 10¹¹ phage of the random 15-mer pIII library. The suspension was first incubated several hours at room temperature, and then placed at 4° C. overnight. The bead was then washed ten times with 15 ml of TTBS, 10 minutes each, with gentle rocking at room temperature. The liquid was carefully aspirated away from the bead. The bound phage were eluted from the bead with Elution Buffer (200 μl 0.1N HCl, adjusted to pH 2.2 which glycine, 1 mg/ml BSA) at room temperature for 5 minutes, gently rocking. The released phage were transferred to a new tube (leaving behind the bead) and neutralized with 0.187 (× volume of Elution Buffer) of 1 M Tris-HCl, pH 9.1. A small amount of the phage was then used to infect E. coli K91Kan cells that were prepared from an overnight culture. Infected cells were plated on LB agar plates containing 40 μg/ml tetracycline. Since the phage carry a tetracycline resistance marker, only infected cells grow on the plates. Cultures were prepared from picked colonies from these plates and grown overnight. The phage were harvested and precipitated twice with PEG for PCR to synthesize the inserts selected in this round one of the panning procedure and to be sequenced. The phage remaining after plating the small amount needed for sequencing were used to generate an unselected E. coli culture and then PEG precipitated two times. The precipitated phage were incubated with a fresh, antibody coated bead to begin round two of the panning procedure as described above. Selected phage identified in the second panning cycle were sequenced, and the third panning process was then performed as described above.

Those phage selected from round three of the panning procedure were sequenced using the following primer set:

Forward: 5′-GTCATTGTCGGCGCAACTATC-3′ (SEQ ID NO:6)

Reverse: 5′-AGGTGTATCACCGTACTCAG-3′ (SEQ ID NO:7)

The peptide sequences of the round three mimotopes of the present invention are disclosed in Table A, representing SEQ ID NOs:1-5. Of the five mimotopes that were sequenced from round three of the panning procedure, some of the sequences were found multiple times. SEQ ID NO:1 was found and sequenced 18 times; SEQ ID NO:2 was found and sequenced 5 times; and SEQ ID NO:3 was found and sequenced 2 times. The remaining mimotope sequences (SEQ ID NOs:4-5) were each found and sequenced one time.

### EXAMPLE 3

### Confirmation of Mimotope Binding Using Surface Plasmon Resonance (i.e., BIACore)

BIACore confirmation of mimotope binding to Mab 2G12 was determined for four of the identified peptide sequences disclosed in the present application. For the first assay, a batch of individually selected phage was prepared from the transduced E. coli cells described in Example 2. The phage were concentrated by PEG precipitation to be used in the assay. Initially, a murine anti-phage pVIII monoclonal antibody (anti-M13) is captured by a BIACore CM5 chip that is coated with a standard rabbit anti-mouse (Fc) antibody (RAMFc). The anti-M13 antibody captures the chimeric phage selected from the panning procedure described in Example 2. Mab 2G12 is then flowed over the chip in order to describe and confirm binding of the antibody to the captured chimeric phage pIII.

Specifically, RAMFc was first immobilized on the gold surface of the BIACore CM5 chip (Uppsala, Sweden) containing dextran by amine coupling. Twenty-five microliters of anti-M13 antibody (Amersham Pharmacia Biotech, Piscataway, N.J.), at 1 mg/ml, was injected into the flowcell containing the chip at a flow rate of 5 μg/min and captured by the immobilized RAMFc. The captured anti-M13 was measured in resonance units (RU). After completing the initial control tests of the flowcell, the chip was regenerated by injecting 10 μl of 20 mM HCL, 3 times, into the flowcell. To determine whether Mab 2G12 can detect the mimotopes isolated in the panning procedure described herein, 25 μl of anti-M13 at 1 mg/ml was first injected into the flowcell. Fifty microliters of the round three phage preparation of the mimotope sequence identified by SEQ ID NO:3, WKIPDHGIVVFSWFP, at >1×10¹⁰ phage particles per ml, was injected into the flowcell. This was followed by 25 μl of Mab 2G12 at 100 μg/ml. The amount of Mab 2G12 captured on the chip was subsequently measured in RUs. This procedure was repeated for the following round three mimotopes: RSGHKVWVVSTKESS (SEQ ID NO:1), KCCFAESSRSGTGRY (SEQ ID NO:2) and VLRLMECHFQCVPSL (SEQ ID NO:4). BIACore analysis confirmed that each of the four mimotopes displayed on phage that were tested indeed bind to the monoclonal antibody 2G12. Of the four mimotopes tested by this procedure, RSGHKVWVVSTKESS (SEQ ID NO:1) bound Mab 2G12 the tightest at 312 RUs. In comparison, KCCFAESSRSGTGRY (SEQ ID NO:2) bound at 120 RUs, WKIPDHGIVVFSWFP (SEQ ID NO:3) bound at 24 RUs, and VLRLMECHFQCVPSL (SEQ ID NO:4) bound at 17 RUs.

Surface plasmon resonance was also used to confirm the binding of synthetic mimotope peptides identified from the panning procedure described in Example 2 to Mab 2G12. In this assay, a solution of biotinylated mimotope peptide is injected into a flowcell containing a BIACore chip coated with streptavidin. The biotin-streptavidin interaction immobilizes the synthetic mimotope peptide on the chip surface. Mab 2G12 is then injected into the flowcell to determine if the antibody recognizes and is captured by the bound mimotope. Specifically, the streptavidin BIACore chip was warmed to room temperature for 30 minutes. The chip was then docked and primed 2× with HBS-EP (0.01M HEPES, pH 7.4, 0.15M NaCl, 3 mM EDTA, 0.005% Surfactant P20) and conditioned for three cycles with 1M NaCl/50 mM NaOH. Each synthesized, biotinylated mimotope peptide was diluted 1:2 in HBS (0.01M HEPES, pH 7.4) to a final concentration of 500 μg/ml. Seventy-five microliters of each mimotope peptide was then injected into the flowcell at a flow rate of 5 μl/min. Mab 2G12 was resuspended in sterile water to a concentration of 0.25 μg/μl, and 100 μl of the antibody was injected into the flowcell containing the captured synthetic peptide at a flow rate of 5 μl/min. The captured Mab2G12 was measure in RUs. Of the mimotopes tested by this procedure, the synthetic peptide represented by SEQ ID NO:3, WKIPDHGIVVFSWFP, bound Mab 2G12 the tightest at 1193 RUs. In comparison, RSGHKVWVVSTKESS (SEQ ID NO:1) bound at 117 RUs, VLRLMECHFQCVPSL (SEQ ID NO:4) bound at 133 RUs, and TLKSLPYRAVLGAQA (SEQ ID NO:5) bound at 37 RUs. The peptide represented by SEQ ID NO:2, KCCFAESSRSGTGRY, showed no binding under these conditions. A negative result, as with the mimotope represented by SEQ ID NO:2, indicates that this particular mimotope does not bind well as a monovalent structure (e.g., a synthetic peptide). Thus, the mimotope represented by SEQ ID NO:2 binds to Mab 2G12 more efficiently as a multivalent structure (e.g., when within the pIII on the tip of the filamentous phage particle).

### EXAMPLE 4

### Extraction and Purification of OMPC

A. First Method

All materials, reagents and equipment are sterilized by filtration, steam autoclave or ethylene oxide, as appropriate. Aseptic technique is used throughout.

A 300 gm (wet weight) aliquot of 0.5% phenol inactivated cell paste of Meningococcal group B11 is suspended in 1200 ml of distilled water by stirring magnetically for 20 minutes at room temperature. The suspended cells are pelleted at 20,000×g for 45 minutes at 5° C.

For extraction, the washed cells are suspended in 1500 ml 0.1 M Tris, 0.01 M EDTA Buffer pH 8.5 with 0.5% sodium deoxycholate (TED Buffer) and homogenized with a 500 ml Sorvall Omnimixer at setting 3 for 60 seconds. The resulting suspension is transferred to ten Erlenmeyer flasks (500 ml) for extraction in a shaking water bath for 15 minutes at 56° C. The extract is centrifuged at 20,000×g for 90 minutes at 5° C. and the viscous supernatant fluid is decanted (volume=1500 ml). The decanted fluid is very turbid and is recentrifuged to clarify further at 20,000×g for 90 minutes at 5° C. The twice spun supernatant fluid is stored at 5° C. The extracted cell pellets are resuspended in 1500 ml TED Buffer. The suspension is extracted for 15 minutes at 56° C. and recentrifuged at 20,000×g for 90 minutes. The supernatant fluids which contained purified OMPC are decanted (volume=1500 ml) and stored at 5° C.

B. Second Method

All material, reagents, equipment and filters are sterilized by heat, filtration or ethylene oxide, except for the K-2 ultracentrifuge which is sanitized with a 0.5% formalin solution. Overnight storage of the protein is at 2-8° C. between steps. A 0.2-micron sterile filtration is conducted just before the final diafiltration to ensure product sterility.

Two 600-liter batches of Neisseria meningitidis are fermented and killed with 0.5% phenol, then concentrated to roughly 25 liters using two 10 ft² 0.2 micron polypropylene cross-flow filtration membranes. The concentrated broth is then diafiltered with 125 L of cell wash buffer (0.11 M NaCl, 17.6 mM sodium phosphate dibasic, 23.3 mM NH₃Cl, 1.34 mM KCl, adjusted to pH 7 with 85% phosphoric acid followed by 2.03 mM magnesium sulfate heptahydrate).

For extraction, an equal volume of 2×-TED buffer (0.2M Tris, 0.02M EDTA adjusted to pH 8.5 with concentrated HCl, followed by the addition of 1.0% sodium deoxycholate) is added to the cell slurry. The resulting slurry is heated to complete the extraction of OMPC from the cells.

For further purification, the extracted cell slurry is centrifuged at 30,000×g (18,000 rpm) in a “one-pass” flow mode in a K-ultracentrifuge, and the supernatant stream is collected. The low-speed supernatant is concentrated to 10 liters on two 0.1-micron polysulfone autoclavable hollow-fiber membranes and collected in an 18 liter sterile bottle. The filtration equipment is given two 4-liter rinses with TED buffer (0.1M Tris, 0.01M EDTA, adjusted to pH 8.5 with concentrated HCl, followed by the addition of sodium deoxycholate to 0.5%) which is combined with the retentate. The retentate is subdivided into two or three equal parts. Each part was centrifuged at 80,000×g (35,000 rpm) for 30 minutes. The OMPC protein is pelleted, and the majority of soluble proteins, nucleic acids and endotoxins remain in the supernatant, which is discarded. The pelleted protein is resuspended by recirculating 55% 5° C. TED buffer through the rotor. The first high-speed resuspensions are combined and subjected to a second low-speed spin. The second low-speed spin ensures that residual cell debris is removed from the product stream. The second low speed supernatant is subdivided into two or three equal parts. Each fraction is given two consecutive high-speed spins. All high-speed spins are operated under the same conditions and each further purified the OMPC protein.

For sterile filtration and final diafiltration, the third high-speed resuspensions are diluted with an equal volume of TED buffer and filtered through a 0.2-micron cellulose acetate filter. When all fractions are permeated, an 8 L TED buffer rinse is used to flush the filtration system. The permeate and rinse are combined and concentrated to 3 liters on a 0.1-micron polysulfone autoclavable hollow-fiber membrane. The material is then diafiltered with 15 L of sterile pyrogen free water. The retentate is collected in a 4-liter bottle along with a 1 L rinse to give the final product. The final aqueous suspension is stored at 2-8° C., as purified OMPC.

C. Third Method

OMPC is purified from 0.2 M LiCl-0.1M Na acetate, pH 5.8 extracts by ultracentrifugation, by the method of Frasch et al., 1974 J. Exp. Med. 140:87-104.

### EXAMPLE 5

### Conjugation of Mimotope to an Immunogenic Carrier, OMPC

The following is an illustration, rather than a limitation, describing how a mimotope of the present invention can be conjugated to an immunogenic carrier, specifically N. meningitidis OMPC.

A. Thiolation of OMPC

OMPC (43.4 mg, 10 ml) is pelleted by ultra-centrifugation (43K rpm, 2 hr, 4° C.). The pellet is resuspended in a sterile filtered (0.22-micron) solution: pH 11, 0.1 M borate buffer (4 ml), N-acetyl homocysteine thiolactone (45 mg), DTT (15 mg), and EDTA (85 mg). The resulting solution is degassed and purged with nitrogen (process repeated 3×) and is stored under N₂ overnight at room temperature. The thiolation mixture is transferred to a centrifuge tube and topped with pH 8.0, 0.1 M phosphate buffer (approximately 4.5 ml). The protein is pelleted via ultra-centrifugation, resuspended in pH 8.0, 0.1 M phosphate buffer, and is repelleted by ultra-centrifugation. This pellet is resuspended in 1× TED buffer, with a total resuspension volume of 7.0 ml.

B. Conjugation

The beta-maleimidopropionyl peptide (5.8 μmol) is dissolved in acetonitrile (1.0 ml) giving Solution P. A solution of beta-maleimidopropionic acid (5.5 μmol) in water (1.0 ml) is prepared, which is Solution M.

Thiolated OMPC (6.0 ml, 5.77 μmol), from Step A, is transferred to a sterile 15 ml centrifuge tube. This solution is vortexed and solution M (420 μl, 2.31 μmol) is added. The mixture is stirred briefly and incubated at room temperature for 10 minutes. Next, the reaction mixture is vortexed and solution P (596 μl, 3.46 μmol) added. The reaction mixture is vortexed briefly and incubated at room temperature for 2 hours.

The conjugate is spun in a clinical centrifuge to remove any precipitated material. The supernatant is removed and the conjugate is pelleted by ultracentrifugation (43K rpm, 2 h, 4° C.). The pellet is resuspended in TED buffer (total volume 6.5 ml), affording mimotope-OMPC conjugate.

1. An antigenic conjugate of HIV-specific mimotope covalently linked to an immunogenic carrier molecule, wherein said conjugate is of the formula
[mimotope(s)]ₙ˜[carrier]
wherein:
mimotope(s) are peptidyl sequences which mimic antigenic epitopes of gp120, said mimotope being a peptide comprising one or more amino acid sequences selected from the group consisting of amino acid sequences of SEQ ID NOs:1-5;
n is 1-200, wherein n is the number of mimotope peptides covalently linked to a carrier;
˜ indicates covalent linkage; and
carrier is an immunogenic molecule to which the mimotope is conjugated.

2. The antigenic conjugate of claim 1, wherein said carrier is a molecule selected from the group consisting of Neisseria meningitidis OMPC particles, HBV-core antigen, HBV-surface antigen, tetanus toxoid, diptheria toxoid, rotavirus VP6, HIV protein gp120, HIV protein gp41, and HIV capsid particles composing p24.

3. The antigenic conjugate of claim 1, wherein said carrier is OMPC of N. meningitidis.

4. The antigenic conjugate of claim 1, wherein the covalent linkage between mimotope and carrier consists essentially of a bigeneric spacer.

5. An isolated mimotope polypeptide comprising one or more amino acid sequences selected from the group consisting of the amino acid sequences of SEQ ID NOs:1-5.

6. A pharmaceutical composition comprising an antigenic conjugate of claim 1, said conjugate mixed with a biologically effective adjuvant, protein, or other agent capable of increasing the immune response, wherein said composition is useful as a vaccine capable of producing specific HIV neutralizing antibodies in mammals.

7. A method of generating an immune response against HIV in an individual comprising administering to the individual an effective amount of the pharmaceutical composition of claim 6.


SEQUENCE LISTING (7 sequences)

SEQ ID NO: 1 - protein, 15 aa, Artificial Sequence
  Synthetic peptide mimotope
RSGHKVWVVSTKESS

SEQ ID NO: 2 - protein, 15 aa, Artificial Sequence
  Synthetic peptide mimotope
KCCFAESSRSGTGRY

SEQ ID NO: 3 - protein, 15 aa, Artificial Sequence
  Synthetic peptide mimotope
WKIPDHGIVVFSWFP

SEQ ID NO: 4 - protein, 15 aa, Artificial Sequence
  Synthetic peptide mimotope
VLRLMECHFQCVPSL

SEQ ID NO: 5 - protein, 15 aa, Artificial Sequence
  Synthetic peptide mimotope
TLKSLPYRAVLGAQA

SEQ ID NO: 6 - DNA, 21 bp, Artificial Sequence
  oligonucleotide
gtcattgtcggcgcaactatc

SEQ ID NO: 7 - DNA, 20 bp, Artificial Sequence
  oligonucleotide
aggtgtatcaccgtactcag